(19) United States
(12) Patent Application Publication
(10) Pub. No.: US 2025/0352106 A1
(43) Pub. Date: Nov. 20, 2025

(54) DRIVER/OPERATOR FATIGUE DETECTION SYSTEM

(71) Applicant: Joshua R&D Technologies, LLC, Plano, TX (US)

(72) Inventor: David Pickett, Plano, TX (US)

(21) Appl. No.: 19/284,606

(22) Filed: Jul. 29, 2025

Related U.S. Application Data
(63) Continuation of application No. 18/375,815, filed on Oct. 2, 2023, now abandoned.

(51) Int. Cl. A61B 5/18; A61B 5/00; A61B 5/1455; A61B 5/375

(52) U.S. Cl. CPC A61B 5/18; A61B 5/1455; A61B 5/375; A61B 5/4809; A61B 5/6803; A61B 5/7405; A61B 2562/0219

(57) ABSTRACT

A driver or operator fatigue detection system may reduce or prevent drivers or operators from entering micro sleep mode and losing control of their responsibilities. Bio feedback from a brain wave sensor placed on a driver's or operator's hatband/headband may sense when the driver or operator is entering micro sleep mode. A loud alarm or other alert may sound when the driver or operator begins to enter micro sleep mode. This may not only awaken the driver/operator, but the loud, unpleasant startling alarm/alert may condition the hind brain to not begin sleep again when driving to avoid the loud punishment. When the system alarms due to an oncoming micro sleep event, the system can cause the driver's/operator's smartphone to call or contact the office/dispatcher to report the incident.

### CROSS-REFERENCE TO RELATED APPLICATIONS

The present application is a continuation of U.S. patent application Ser. No. 18/375,815 filed Oct. 2, 2023, which is a non-provisional of, and claims priority to, U.S. Patent Application No. 63/378,040, filed Sep. 30, 2022, the disclosures of which are incorporated by reference in their entirety.

### FIELD OF THE DISCLOSURE

The present disclosure generally relates to sensing bio feedback to determine if a person is beginning to enter micro sleep. Microsleep is a fleeting, uncontrollable, brief episode of sleep which can last anywhere from a fraction of a second up to 10 seconds. Episodes of microsleep occur most frequently when a sleepy person is trying to fight sleep and remain awake. They may occur without warning while driving or operating equipment and increase the risk of serious accident.

### BACKGROUND

Operators of vehicles, equipment, aircraft, machinery, sentries, watchmen, monitors and air traffic controllers who unexpectedly fall asleep can cause serious accidents with loss of life and property. Entering just into temporary micro sleep can have devastating consequences for drivers, pilots, security and military personnel, observers, monitors, and people in many types of activities where being alert is critical. The most dangerous instant is when the operator just begins micro sleep; the time just before the eyes begin to close and the operator loses awareness and just has no control. At this point, video cameras monitoring the eyes/eyelids and motion detectors will not detect that the operator is substantially unconscious and unable to operate the vehicle or his duties. For example, many truck accidents result from operator fatigue, and most highway deaths involve trucks. Even a gentle roll over onto soft shoulder grass with no crash damage or collision, can cost $250,000 or even more. This will increase operator's insurance costs and expensive deductibles. Even a roll over with no crash damage or collision can increase operator's insurance costs and result in expensive deductibles. Further, injuries from truck collisions can result in millions of dollars in cost, if not death, plus the human factor of pain and suffering and loss. Injuries also can lead to bad press for the operator's image and damages the ability to increase or sustain business volume. Sleep detectors that work by alarming when the chin hits the chest or upon driver “lack of motion” or eyes closing are detected too late in vehicle operations particularly; when such lack of motion alarm sounds, the vehicle may already be heading off the road and recovery may not be probable.

### SUMMARY

Embodiments of the present disclosure may provide a driver/operator fatigue detection system that may reduce or prevent drivers or operators of equipment or attendants to activities from entering micro sleep mode and subsequently into sleep and unconsciousness. Bio feedback from a brain wave sensor placed on a driver's hatband/headband may detect a characteristic brain wave of a person entering micro sleep mode. A loud alarm or other alert may sound when the driver or operator begins to enter micro sleep mode. This may not only awaken the driver or operator, but the loud, unpleasant startling alarm/alert may condition the driver's or operator's hind brain to not begin sleep again when driving/operating to avoid the loud punishment.

Bio sensors may be mounted in a headband, hatband, or other mechanism to connect with the driver's head area. The headband, hatband, or other mechanism with bio sensors may be connected to a small controller device and/or a driver's smartphone or tablet by way of Bluetooth or wireless or hardwire technology. The controller device may have a large memory to record any events and the truck/vehicle position and speed, such as every 5 minutes over a specified time frame, such as a month at a time, and record all micro sleep incidents associated with the driver or operator. However, it should be appreciated that the events, position, and/or speed may be recorded more or less often without departing from the present disclosure.

When the system alarms due to an oncoming micro sleep event, the system can cause the driver's smartphone to call or otherwise contact the office/dispatcher to report the incident. The driver's location and speed also may be reported. The driver's dispatcher may call the driver and instruct the driver to pull off at the next rest stop, take a nap, and then call for further instructions. The system may optionally sound the horn, flash lights, set warning flashers, report and record many other parameters including, but not limited to, location, speed, fuel level, engine, cab, and outside temperature, and/or cargo temperature. Any of these parameters may be downloaded by the office via the driver's smartphone or other wireless device. The system may turn the truck engine off or start it, set the brakes, lock/unlock doors, sound horn, lights on and off, and may host a live microphone and multiple live video cameras. Other sensors may be provided in embodiments of the present disclosure.

Other embodiments of the present disclosure may provide an operator fatigue system comprising: a headband having a plurality of EEG brain sensors capable of providing real-time feedback on one or more items selected from the following: brain activity, heart rate, breathing, and body movements; and a mobile device connected to the headband through Bluetooth or wireless transmission, wherein the mobile device may receive the real-time feedback and use a multi-channel EEG machine learning model to classify the operator's brain wave patterns to determine whether they are alert to operate a vehicle. The plurality of EEG brain sensors may comprise two sensors positioned on a forehead of the operator, two sensors positioned behind the ears of the operator, and three reference sensors. The two sensors positioned behind the ears of the operator may be gyroscope and accelerometer body sensors. The three reference sensors may be PPH and pulse oximetry breath and heart sensors. The sensors may detect and measure passively without use of electrical stimulation. The headband also may include adjustable arms to adjust a length of the headband to a head of the operator. Vehicle dynamics, operator notes, and video operation of the operator and direction of travel may be transmitted along with EEG data to the mobile device. The operator notes may comprise alertness and driving metrics to classify a state of the operator. The EEG data may be correlated to the state of the operator to produce the multi-channel EEG machine learning model. A recurrent neural network (RNN) utilizing long short-term memory (LSTM) may be used to produce the multi-channel EEG machine learning model.

Further embodiments of the present disclosure may comprise operator fatigue system comprising: a four-channel BLE-enabled EEG headset housed in a ballcap and worn while the operator is driving, the headset capable of providing real-time feedback on one or more items selected from the following: brain activity, heart rate, breathing, and body movements; and a mobile device that may collect EEG data collected by the headset and transmitted to the mobile device through wireless transmission, wherein the collected EEG data may be used to develop a data and image processing script to classify whether the operator is fatigued. Vehicle dynamics, operator notes, and video operation of the operator and direction of travel may be transmitted along with the EEG data to the mobile device. When the operator is classified as fatigued, an audible alert may be provided to the operator. When the audible alert is not acted upon within a specified period of time, a communication may be initiated to get the operator's attention to stop driving. When the operator is classified as fatigued, the headset may be activated to electro stimulate the operator's brain.

Further embodiments of the present disclosure may provide an operator fatigue system to prevent the operator from entering micro sleep mode comprising: a headband positioned on the operator with a plurality of brain wave sensors that sense when the operator is entering micro sleep mode; a mobile device in communication with the headband to receive EEG data from the plurality of brain wave sensors; and an alert system activated by the mobile device when the plurality of brain wave sensors sense when the operator is entering micro sleep mode. The alert system may be an audible alert to awaken the operator and condition a hind brain of the operator to not begin sleep again. The alert system may cause the mobile phone to provide a report to a third party about the micro sleep mode incident. When the operator enters micro sleep mode, the headset may be activated to electro stimulate the operator's brain. Vehicle dynamics, operator notes, and video operation of the operator and direction of travel may be transmitted along with EEG data to the mobile device.

### BRIEF DESCRIPTION OF THE DRAWINGS

For a more complete understanding of this disclosure, reference is now made to the following description, taken in conjunction with the accompanying drawings, in which:

FIG. 1 depicts a headband with sensors for a driver fatigue detection system according to an embodiment of the present disclosure; and

FIG. 2 depicts a driver fatigue detection system according to an embodiment of the present disclosure.

### DETAILED DESCRIPTION

Embodiments of the present disclosure may provide a driver or operator fatigue detection system which may include a bio feedback device where sensor(s) may be attached to a person, such as on the head, with a cord/cable leading to the plug on a smartphone or other computing device (or wirelessly via Bluetooth or other similar transmission method) wherein the smartphone may provide the computing power and memory for the driver fatigue system operation and also can communicate through text or cell phone to an outside recipient such as a dispatcher.

When a driver (operator/wearer) begins to enter micro sleep, the system may detect the unique entering-microsleep brain wave pattern and sound a “shattering” alarm or otherwise provide an alert, awaking the driver/operator quickly enough where control lost just as micro sleep is entered can be regained “in time.” The system may detect when an individual just begins to enter sleep condition (micro sleep) and before eyes close and/or the head “nods off.” The alarm may awaken the wearer of the system. At the same time, or a preset time later, additional actions may be initiated such as sensory awake alarm escalation, shutting down equipment, local alert alarms and reporting to others who may take various actions to awake the sleeper or mitigate the situation by shutting down machinery, setting warning alarms, taking remote control, sending an alert to a dispatcher, and/or literally dialing 911.

Sleep/micro sleep incidents may be recorded in the driver's smartphone memory for download later along with time, date, exact location, speed, and/or other parameters which can also be used for accident/incident analysis. The driver's office or dispatcher could be called to immediately report the incident so they can take preventative and corrective action. The detection system may use the smartphone's motion detection and accelerometers to sense an accident while it is happening and report, through phone or text, to a dispatcher or other party. The system may be interfaced with other devices, such as computers or tablets or the vehicle's on-board computer, for downloading incident data for recording and even alert operators of warnings or danger from the vehicle's computer.

The very loud, unpleasant, shattering wake up alarm on the detection system may, in a fairly short period, possibly weeks, train the operator's “hind brain” that he/she may not start to fall asleep while driving or he/she may be painfully startled by the “shattering” loud wake up alarm or alert. This may be akin to the “Pavlov Dog” reaction where a dog begins to salivate when the can opener is operated.

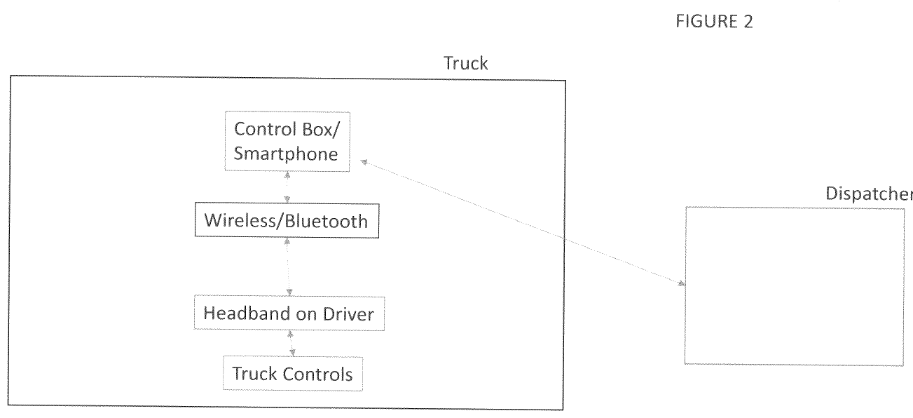

The detection system according to embodiments of the present disclosure, such as depicted in FIG. 2, may be powered by a smartphone which provides substantial computing power, memory, a cell phone, accelerometer (motion detection), GPS location, and/or camera (as a verification opportunity). Connected Bluetooth or wireless communication/transmission may even play music and audio books, among performing other functions in embodiments of the present disclosure. Bluetooth or wireless communication/transmission, when part of the driver startle alarm, can also control the truck's horn and flashing lights; invaluable in alerting others around the truck about the driver falling asleep. The smartphone's Bluetooth or wireless communication/transmission may even remotely shut down the engine, set the brakes, flash lights, sound the horn, lock/unlock doors, and/or perform other functions to improve safety related to the truck when the driver has been detected as entering micro sleep mode. The detection system also may have the ability to provide the operator's office or dispatcher with information from the vehicle's computer including, but not limited to, speed, location, fuel level, outside and inside temperature and even continuous video and audio feed of the driver, and/or road in embodiments of the present disclosure.

In addition to startling the driver awake, the detection system's built-in cell phone may immediately call the driver's office or dispatcher to report the sleep incident so the office or dispatcher may act, such as instructing the driver to swap out with his co-driver, pull over to sleep, stop at the next truck stop, or other opportunity, among other instructions. The smartphone's extensive memory may store exact time and coordinates while the detection system is being worn, preventing driver from removing the headband of the detection system when he is supposed to be wearing it. The detection system may be mounted where the camera monitors the driver/face. The smartphone may detect if it is mounted properly and that it is sending actual operator's own bio feedback. Lack of recording bio feedback may initiate a call or text notice to the office or monitor. The detection system may even detect which driver is wearing it after it learns the individual driver's bio feedback profile. Due to its bio feedback design, the detection system according to embodiments of the present disclosure may be generally free from “falsing”—that is, false alarms triggered by incorrect input such as head nodding, eyelids closed, and/or lack of movement that render other systems unreliable and insensitive to the actually entering into micro sleep event.

The brain waves of the operator may be monitored and the brain wave pattern where the operator just begins to enter micro sleep may be profiled. Numerous entering micro sleep brain wave patterns may be recorded for an individual driver and a common signature of this bio sensor event may be determined for that operator. The entering micro sleep brain wave signature of numerous operators may be determined and then the entire data set compared and analyzed to determine a common brainwave pattern for entering micro sleep common to the test subject's entire population. Next, all the original test simulator operators may be fitted with the detection system programmed to detect the above determined population unique brain wave pattern determined above to detect the operator entering micro sleep. If the unique brain wave profile for entering micro sleep discovered above is determined to be detected entering micro sleep for the entire initial population, another population of different simulator drivers will wear the detection system and subjected to the simulator or live boring sleep-inducing driving to determine if the above determined unique entering micro sleep brain wave pattern is also applicable to individuals in a comparative group. While new subjects—not from the initial population—are in the driving simulator and the detection system may be watching/detecting for the unique entering micro sleep brain wave pattern in order to alarm the operator of entering micro sleep, the detection system also may be recording the subject's brain waves at that time to increase the data population of entering micro sleep, further learning, confirming and fine tuning the key unique entering micro sleep brain wave pattern. Next, a population may be tested in the simulator under the same conditions that encourage falling asleep, various distractions will be introduced to assure that the detection system may still detect entering micro sleep despite driving distractions such rainstorms, heavy traffic, difficult roads, rough roads, (radio) music, and/or conversation of another driver. This may ensure that the system may detect the unique entering micro sleep brain wave pattern entering micro sleep despite various distractions.

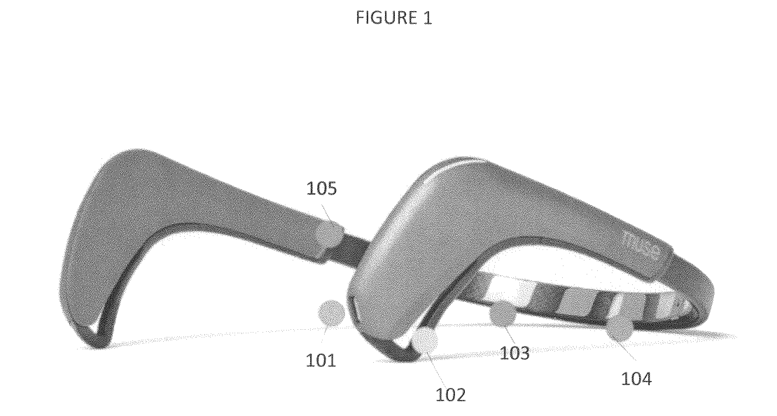

FIG. 1 depicts a headband with sensors that may be used for data collection according to an embodiment of the present disclosure. In an embodiment of the present disclosure, a Muse 2 headband may be used. Muse 2 is a multi-sensor device that may provide real-time feedback on one or more items including, but not limited to, brain activity (i.e., real-time brainwave feedback through EEG), heart rate, breathing, and/or body movements. The headband may include 7 EEG brain sensors including, but not limited to, 2 on the forehead 104, 2 behind the ears 102 (which may be gyroscope and accelerometer body sensors), and 3 reference sensors 103 (which may be PPH and pulse oximetry breath and heart sensors). However, more or fewer sensors may be utilized on a headband according to an embodiment of the present disclosure. Similarly, the sensors may be set in different positions without departing from the present disclosure. Regardless the number or positioning of the brain sensors, the sensors may detect and measure the activity of the wearer's brain. Such detection and measurement may be done passively (i.e., without use of electrical stimulation). The headband may connect to a mobile device through Bluetooth or other wireless or hardwire communication method. The connection may be through micro-USB charging port 101 in an embodiment of the present disclosure. The headband also may include adjustable arms in an embodiment of the present disclosure. The adjustable arms and/or an elastic section at a back portion of the headband may allow the headband to fit a variety of heads by adjusting the headband's length. This adjustment may allow the headband to fit properly to ensure that the sensors have strong signal quality.

A multi-channel EEG machine learning model may be used to classify a driver's brain wave patterns to determine whether they are alert to operate a motor vehicle. In embodiments of the present disclosure, a standard four-channel EEG may be used, and training data may be recorded from various drivers in a simulated driving session for extended hours in various reset states. A trained observer may journal their observations based upon alertness as well as driving metrics, such as the following: speed control, if not using cruise control, lane departure, and/or lane position (i.e., vehicle sway). This data may be used to classify the state of the driver. At which point, the collected driver EEG waveforms may be correlated to the classified state of the driver to produce a trained model. Deep learning and, specifically, an RNN (Recurrent Neural Network) utilizing LSTM (Long Short-Term Memory) may be used in embodiments of the present disclosure.

Overall accuracy for classification validation is approximately 75% with the latest model. However, as more training information and simulation resources are used, the accuracy may be improved. For example, the training data set may be increased in controlled observation in a driving simulation lab and live collected data from the target audience, which may be long haul truckers in an embodiment of the present disclosure.

The wearable device utilized in embodiments of the present disclosure may be a four-channel BLE-enabled EEG headset housed in a ballcap to stream the EEG data to a smartphone or tablet for collection. The data, along with vehicle dynamics (GPS, acceleration, and/or speed), the truck driver's notes, and video observation of the driver and direction of travel, may be collected. In addition, a data and image processing script may be developed to classify each test for the driver based on similar conditions mentioned above. 75% of these results, both from simulations and actual observed driving conditions, may be used to train a new RNN model. The remaining 25% of the results may be used for model validation. It is estimated that approximately 1,500 hours of driving data collected over approximately 50 drivers may provide sufficient data for an accurate model.

The detection system may be utilized in conjunction with a smartphone or tablet. The driver may wear the headband or hatband associated with the detection system while driving. For example, the ML model may determine that the driver is fatigued. In that case, an audible alert may be provided, and if not acted upon, a phone call or other communication will be initiated to attempt to get the driver's attention and have them pull over and reset for some time. The wearable headband may be used to harmlessly electro stimulate the driver's/operator's brain, to startle the driver/operator awake.

The detection system according to embodiments of the present disclosure is a bio feedback wearable head band device with sensors attached to the head, connected via Bluetooth to a smartphone that provides ample computing power to run an application that will identify the documented signature brain pattern to detect entry into microsleep even before eyes close and driver/operator loses control. When a driver (operator/wearer) begins to enter micro sleep, the system may detect the signature brain wave pattern unique to beginning to fall asleep and will sound a ‘shattering’ alarm, waking the driver/operator/sentry quickly enough to bring full consciousness for control to be regained “in time”. Such startling, unpleasant ‘conditioning’ will train the operators' “hind brains” to avoid microsleep entry to avoid the painful alarm. The phone accelerometers may detect accidents and report the GPS location to dispatchers. There may be a 911 voice capability. The system may detect an unauthorized wearer using fingerprint-type wearer identification and may detect intoxication or other medical issues.

An application (App) may be provided for standard smartphones linked to off-the-shelf sensor head bands to sound an alarm upon detection of the signature microsleep brain wave pattern and may also instantly identify patterns such as intoxication, health conditions and other indications that need to be reported via the smartphone's cellular connection to a dispatcher/controller to ensure operator safety. “Falsing” would be unlikely with such technology. Such a system may save countless lives from fatigue and sleep related incidents/accidents in trucking, transportation, air traffic control, flight operations, machine operation and anywhere falling asleep is hazardous/dangerous, such as sentries and security.

Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. An operator fatigue system comprising:
a headband having a plurality of EEG brain sensors capable of providing real-time feedback on one or more items selected from the following: brain activity, heart rate, breathing, and body movements; and
a mobile device connected to the headband through Bluetooth or wireless transmission, wherein the mobile device receives the real-time feedback and uses a multi-channel EEG machine learning model to classify the operator's brain wave patterns to determine whether they are alert to operate a vehicle.

2. The operator fatigue system of claim 1, the plurality of EEG brain sensors comprising:
two sensors positioned on a forehead of the operator, two sensors positioned behind the ears of the operator, and three reference sensors.

3. The operator fatigue system of claim 2, wherein the two sensors positioned behind the ears of the operator are gyroscope and accelerometer body sensors.

4. The operator fatigue system of claim 2, wherein the three reference sensors are PPH and pulse oximetry breath and heart sensors.

5. The operator fatigue system of claim 1, wherein the sensors detect and measure passively without use of electrical stimulation.

6. The operator fatigue system of claim 1, the headband further comprising:
adjustable arms to adjust a length of the headband to a head of the operator.

7. The operator fatigue system of claim 1, wherein vehicle dynamics, operator notes, and video operation of the operator and direction of travel are transmitted along with EEG data to the mobile device.

8. The operator fatigue system of claim 7, wherein the operator notes comprise alertness and driving metrics to classify a state of the operator.

9. The operator fatigue system of claim 8, wherein EEG data is correlated to the state of the operator to produce the multi-channel EEG machine learning model.

10. The operator fatigue system of claim 9, wherein a recurrent neural network (RNN) utilizing long short-term memory (LSTM) is used to produce the multi-channel EEG machine learning model.

11. An operator fatigue system comprising:
a four-channel BLE-enabled EEG headset housed in a ballcap and worn while the operator is driving, the headset capable of providing real-time feedback on one or more items selected from the following: brain activity, heart rate, breathing, and body movements; and
a mobile device that collects EEG data collected by the headset and transmitted to the mobile device through wireless transmission, wherein the collected EEG data is used to develop a data and image processing script to classify whether the operator is fatigued.

12. The operator fatigue system of claim 11, wherein vehicle dynamics, operator notes, and video operation of the operator and direction of travel are transmitted along with the EEG data to the mobile device.

13. The operator fatigue system of claim 11, wherein when the operator is classified as fatigued, an audible alert is provided to the operator.

14. The operator fatigue system of claim 12, wherein when the audible alert is not acted upon within a specified period of time, a communication is initiated to get the operator's attention to stop driving.

15. The operator fatigue system of claim 11, wherein when the operator is classified as fatigued, the headset is activated to electro stimulate the operator's brain.

16. An operator fatigue system to prevent the operator from entering micro sleep mode comprising:
a headband positioned on the operator with a plurality of brain wave sensors that sense when the operator is entering micro sleep mode;
a mobile device in communication with the headband to receive EEG data from the plurality of brain wave sensors; and
an alert system activated by the mobile device when the plurality of brain wave sensors sense when the operator is entering micro sleep mode.

17. The operator fatigue system of claim 16, wherein the alert system is an audible alert to awaken the operator and condition a hind brain of the operator to not begin sleep again.

18. The operator fatigue system of claim 16, wherein the alert system causes the mobile phone to provide a report to a third party about the micro sleep mode incident.

19. The operator fatigue system of claim 16, wherein when the operator enters micro sleep mode, the headset is activated to electro stimulate the operator's brain.

20. The operator fatigue system of claim 16, wherein vehicle dynamics, operator notes, and video operation of the operator and direction of travel are transmitted along with EEG data to the mobile device.
